(19) United States
(12) Patent Application Publication
(10) Pub. No.: US 2015/0377832 A1
(43) Pub. Date: Dec. 31, 2015

(54) Methods, Systems, and Devices for Determining a Magnet Characteristic

(71) Applicants: Wilson Fung, Macquarie University (AU); Harry Xiao, Macquarie University (AU); Josef Oscar, Macquarie University (AU)

(72) Inventors: Wilson Fung, Macquarie University (AU); Harry Xiao, Macquarie University (AU); Josef Oscar, Macquarie University (AU)

(21) Appl. No.: 14/318,404

(22) Filed: Jun. 27, 2014

(51) Int. Cl. G01N 27/72; A61N 1/372; A61N 1/36

(52) U.S. Cl. CPC G01N 27/72; A61N 1/36032; A61N 1/372

(57) ABSTRACT

Disclosed herein are methods, systems, and devices for determining a recommended magnet for an external unit of an implantable medical device. An example diagnostic device includes a primary sensor configured to measure a characteristic of a magnetic field generated by a magnet included in an implantable unit of an implantable medical device. The diagnostic device also includes an output component and a processor component. The processor component is configured to receive one or more measurement signals from the primary sensor. Each measurement signal includes information indicative of the measured characteristic. The processor is also configured to, based on the measured characteristic included in each of the one or more measurement signals, determine a magnet characteristic. The processor component is further configured to cause the output component to output information indicative of the magnet characteristic.

### BACKGROUND

Individuals with certain medical conditions may benefit from the use of an implantable medical device. For example, individuals who suffer from certain types of hearing loss may benefit from the use of a hearing prosthesis. Depending on the type and the severity of the hearing loss, a recipient can employ a hearing prosthesis to assist the recipient in perceiving at least a portion of a sound.

A partially implantable medical device typically includes an external unit that performs at least some processing functions and an implanted component that at least delivers a stimulus to a body part. In the case of a hearing prosthesis, the body part is often in an auditory pathway of the recipient. The auditory pathway includes a cochlea, an auditory nerve, a region of the recipient's brain, or any other body part that contributes to the perception of sound. In the case of a totally implantable medical device, the implanted component includes both processing and stimulation components.

For some implantable medical devices, including some that are totally implantable, the external unit also provides power to the implantable unit. In these devices, the external unit transmits a power signal to the implantable unit via a transcutaneous link or a percutaneous link. To facilitate transmission of the power signal and utility to the recipient, the external unit and the implanted component are often magnetically coupled, with the recipient wearing the external unit on or in close proximity to the recipient's body.

### SUMMARY

A diagnostic device is disclosed. The diagnostic device includes a primary sensor configured to measure a characteristic of a magnetic field generated by an implanted magnet, with the implanted magnet being implanted in a recipient. The diagnostic device also includes an output component and a processor component. The processor component is configured to receive, from the primary sensor, one or more measurement signals from the primary sensor. Each measurement signal includes information indicative of a measured characteristic of the magnetic field. The processor component is also configured to, based on the measured characteristic included in each of the one or more measurement signals, determine a magnet characteristic. The magnet characteristic is indicative of a characteristic of an external magnet to include in an external apparatus that is configured to be magnetically attached to the recipient. The processor is further configured to cause the output component to output information indicative of the magnet characteristic.

A method is also disclosed. The method includes receiving, by a component of a diagnostic device, a measurement indicative of a characteristic of a magnetic field generated by an implanted magnet, with the implanted magnet being included in an implantable unit of an implantable medical device. The method also includes determining, based on the measurement, a magnet characteristic for an external unit of the implantable medical device. The method additionally includes generating an output that includes information indicative of the magnet characteristic.

Additionally, a system is disclosed. The system includes a sensor device and a computing device. The sensor device is configured to measure a magnetic field generated by an implanted magnet included in an implantable medical device. The computing device is configured to receive a plurality of signals from the sensor device, with each signal including information indicative of the measured magnetic field. The computing device is also configured to make a determination, based on a first set of one or more signals included in the plurality of signals, of whether the sensor device is aligned or misaligned over the implanted magnet. If the determination is that the sensor device is aligned, the computing device is configured to (i) determine, based on a second set of one or more signals included in the plurality of signals, a magnet characteristic for a magnet included in an external unit of the implantable medical device, and (ii) generate a first output that includes information indicative of the magnet characteristic. If the determination is that the device is misaligned, then the computing device is configured to (i) determine, from at least two signals included the plurality of signals, a movement that will more closely align the sensor device over the implanted magnet, and (ii) generate a second output that includes information indicative of the movement.

These as well as other aspects and advantages will become apparent to those of ordinary skill in the art by reading the following detailed description, with reference where appropriate to the accompanying drawings. Further, it is understood that this summary is merely an example and is not intended to limit the scope of the invention as claimed.

### BRIEF DESCRIPTION OF THE FIGURES

Various embodiments are described below in conjunction with the appended drawing figures, wherein like reference numerals refer to like elements in the various figures, and wherein:

FIG. 1 is a conceptual diagram illustrating the spatial relationship between components of a medical device, according to an example;

FIG. 2 is a simplified block diagram of a diagnostic device, according to an example;

FIGS. 3A, 3B, and 3C illustrate example arrangements of sensors of the diagnostic device depicted in FIG. 2;

FIG. 4 is a top view of the diagnostic device depicted in FIG. 2, according to an example;

FIG. 5 is a flow diagram of a method for aligning a diagnostic device over a magnet of an implantable unit of a medical device, according to an example;

FIGS. 6A, 6B, and 6C show aspects of the diagnostic device depicted in FIG. 4 based on a proximity to an implantable magnet described in FIG. 1, according to examples;

FIG. 7 is a flow diagram of a method for identifying a magnet characteristic, according to an example;

FIG. 8 is a conceptual diagram of a diagnostic system, according to an example; and

FIG. 9 is a simplified block diagram of a diagnostic device depicted in the diagnostic system of FIG. 8, according to an example.

### DETAILED DESCRIPTION

The following detailed description describes various features, functions, and attributes of the disclosed systems, methods, and devices with reference to the accompanying figures. In the figures, similar symbols typically identify similar components, unless context dictates otherwise. The illustrative embodiments described herein are not meant to be limiting. It will be readily understood that the aspects of the present disclosure, as generally described herein, and illustrated in the figures, can be arranged, substituted, combined, separated, and designed in a wide variety of different configurations, all of which are contemplated herein.

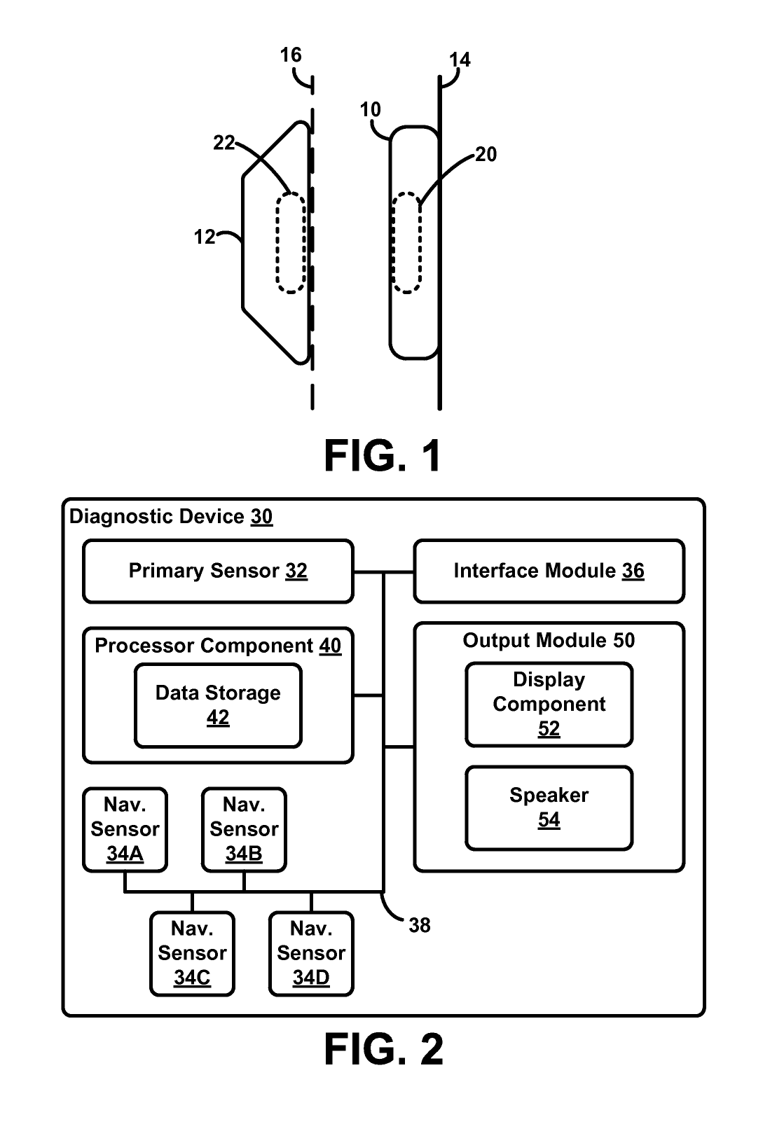

FIG. 1 is a conceptual diagram illustrating the spatial relationship between components of a medical device. For illustrative purposes, the medical device is described herein as a hearing prosthesis, such as a cochlear implant, a bone conduction device, a middle ear implant, or any other hearing prosthesis now known or later developed that is configured to deliver a stimulus to a body part in an auditory pathway up a recipient in order to allow the recipient perceive at least a portion of a sound. In other examples, the medical device may be a different medical device, such as, perhaps, an ocular implant.

The hearing prosthesis includes an implantable unit 10 and an external unit 12. The implantable unit 10 is implanted in a recipient's body and anchored to the recipient's skull 14. The recipient wears the external unit 12, with the external unit 12 typically placed on, or in close proximity to, the recipient's skin 16 at a position that is substantially over the implantable unit 10.

The implantable unit 10 receives at least a power signal from the external unit 12 and generates a stimulus that causes the recipient perceive at least a portion of a sound. In an example in which the hearing prosthesis is a totally implantable hearing prosthesis, such as a totally implantable cochlear implant, the implantable unit 10 receives and processes a sound to generate the stimulus. Alternatively, in an example in which the hearing prosthesis is a partially or mostly implantable hearing prosthesis, the implantable unit 10 generates stimuli based on stimulation signals transmitted by the external unit 12. In some examples, the implantable unit 10 transmits telemetry signals to the external unit 12, which may be interleaved with transmissions of the stimulation signals.

The implantable unit 10 and the external unit 12 communicate via a transcutaneous link. For instance, the implantable unit 10 and the external unit 12 communicate by inductively transmitting signals. In a further example, the external unit 12 modulates the power signal with stimulation signals, thereby facilitating concurrent transmission of both the power signal and the stimulation signals to the implantable unit 10. In another example, however, the implantable unit 10 and the external unit 12 communicate via one or more different and/or additional transcutaneous links or, perhaps, via one or more percutaneous links.

To maintain the external unit 12 in position over the implantable unit 10, the implantable unit 10 and the external unit 12 are magnetically coupled. To this end, the implantable unit 10 includes an implanted magnet 20, and the external unit 12 includes an external magnet 22.

Whether the implanted unit 10 and the external unit 12 are properly coupled depends the combination of the magnets 20, 22. If the retention force of the magnets 20, 22 is too weak, the external unit 12 will not be sufficiently secured to the recipient's body. If the external unit 12 is loosely coupled to the implantable unit 10, the external unit 12 can move when the recipient moves. This can lead to discomfort and/or, in some situations, chafing of the recipient's skin 16, which may discourage the recipient from using the implantable medical device. Moreover, certain motions may cause the external unit 12 to detach from the recipient's skin 16, thereby frustrating the recipient's use of the implantable medical device. The external unit 12 may also be damaged as a result of falling off of the recipient's skin 16, thereby further impairing the recipient's ability to utilize, and benefit from, the implantable medical device.

On the other hand, if the retention force of the magnets 20, 22 is too strong, the force exerted on the recipient's skin 16 may also cause discomfort, which can also discourage the recipient's use of the implantable medical device. In extreme circumstances, a strong attractive force can damage the skin 16 and/or the soft tissue around the implantable unit 10, thereby causing a potentially serious health concern for the recipient.

Accordingly, it is important to select the proper combination of the magnets 20, 22 to adequately retain the external unit 12 to the recipient's skin 16 without causing discomfort, or worse, to the recipient. Because the implanted magnet 20 is not readily accessible post-implantation, one goal may be to select the proper magnet for the external unit 12. In one example, a clinician selects the external magnet 22 from a plurality of magnets. In another example, the external magnet 22 is an electromagnet. In this example, the clinician determines a setting, such as a current, used to drive the magnetic field generated by the external magnet 22 that will provide the proper retention force when the external unit 12 is coupled to the implanted unit 10.

Although the identity or strength of the external magnet 22 can be predicted pre-operatively, a number of factors—such as the amount and type of tissue between the implanted magnet 20 and the external magnet 22 and/or the amount, density, and thickness of the recipient's hair over the implanted magnet 20—can influence the retention force of the magnets 20, 22.

Because of these variables, a clinician may select a less-than-optimal magnet for the recipient in a post-operative setting (i.e., in the operating room or hospital shortly after implantation). Further, if the recipient is not accustomed to wearing the external unit 12, the recipient may not realize that the external unit 12 is too loose or too tight until well after the recipient has been discharged. Thus, if the wrong magnet is placed in the external unit 12, the recipient may need to return to the clinician at a later date, which may be inconvenient to the recipient and, for the aforementioned reasons, limit the recipient's ability, or desire, to use the implantable medical device.

To assist a clinician in identifying a recommended magnet or a characteristic of a recommended magnet, such as a recommended strength of an electromagnet, for the external unit 12 shortly after implantation, a clinician can use a diagnostic device. The diagnostic device may assist the clinician by locating the center of the magnet field of the implanted magnet 12, and by determining a magnet characteristic. The magnet characteristic may help the clinician identify the proper magnet for the recipient in a post-operative setting, especially if the clinician has limited experience. Moreover, properly identifying the external magnet 22 (or the proper strength if the external magnet 22 is an electromagnet) may allow the recipient to acclimate more quickly to the implantable medical device, thereby increasing the likelihood of the recipient using the implantable medical device 10.

FIG. 2 is a simplified block diagram of an example diagnostic device 30 configured to determine a magnet characteristic and to provide an indication of the determined characteristic to a clinician. The example diagnostic device 30 includes a primary sensor 32, navigation sensors 34A-34D, an interface module 36, a processor component 40, and an output module 50, all of which are connected directly or indirectly via circuitry 38.

In an example use, the clinician places the diagnostic device 30 on, or in close proximity to the recipient's skin 16, substantially over the implantable unit 10. The diagnostic device provides visual and/or audio cues to the clinician in order to assist the clinician in aligning the diagnostic device 30 over the implanted magnet 20. Once aligned, the diagnostic device 30 measures the magnetic field of the implanted magnet 20 and, based on the measured magnetic field, determines a magnet characteristic. The magnet characteristic may include a strength of the external magnet 22 or a polarity of the external magnet 22 that will provide the proper retention force when the external unit 12 is coupled to the implanted unit 10. In one example, the magnet characteristic may be an identity of a recommended magnet (e.g., part number or proprietary designator for a magnet) to use as the external magnet 22. The diagnostic device 30 then provides information indicative of the determined magnet characteristic via a visual output and/or an audible output.

In a representative implementation, the diagnostic device 30 is portable and handheld. To this end, the diagnostic device 30 includes an internal power supply (not shown), such as a rechargeable battery. Alternatively, the diagnostic device 30 receives power from one or more replaceable batteries.

The primary sensor 32 is configured to measure a characteristic of the magnetic field of the implanted magnet 20, such as a strength of the magnetic field or a polarity of the magnetic field. The primary sensor 32 then sends a measurement signal, which includes information indicative of measured characteristic, to the processor component 40.

In one example, the primary sensor 32 is a magnetic field sensor, such as magnetometer, that measures a vector of the magnetic field. In this example, the primary sensor 32 includes in the measurement signal information indicative of the measured vector of magnetic field.

In another example, the primary sensor 32 is a magnetic field sensor, such as a Hall Effect sensor, that measures the strength of the magnetic field. In this example, the primary sensor 32 includes in the measurement signal information indicative of the measured strength of the magnetic field.

In an alternative example, the primary sensor 32 includes multiple magnetic field sensors, each of which measures the strength of the magnetic field. In this example, the primary sensor 32 may include a component configured to determine the vector of the magnetic field based on measurements taken by the magnetic field sensors, and the primary sensor 32 includes in the measurement signal information indicative of the determined vector. Alternatively, the primary sensor 32 may send to the processor component 40 multiple measurement signals, each of which includes an indication of a measured strength of the magnetic field.

In yet another example, the primary sensor 32 includes a magnetic object and a mechanical sensor. The primary sensor 32 uses the mechanical sensor to measure the force imparted on the magnetic object by the magnetic field, and primary sensor 32 includes in the measurement signal information indicative of the measured force. In this example, the mechanical sensor is a piezo-pressure sensor, a strain gauge, a load cell, or any other mechanical sensor or combination of mechanical sensors now known or later developed that is suitable for measuring the force imparted on the magnetic object by the magnetic field. In still another example, the primary sensor 32 is any sensor or combination of sensors now known or later developed that is suitable for measuring a characteristic of the magnetic field generated by the implanted magnet 20.

The navigation sensors 34A-3D function to measure a strength of the magnetic field and generate a navigation signal.

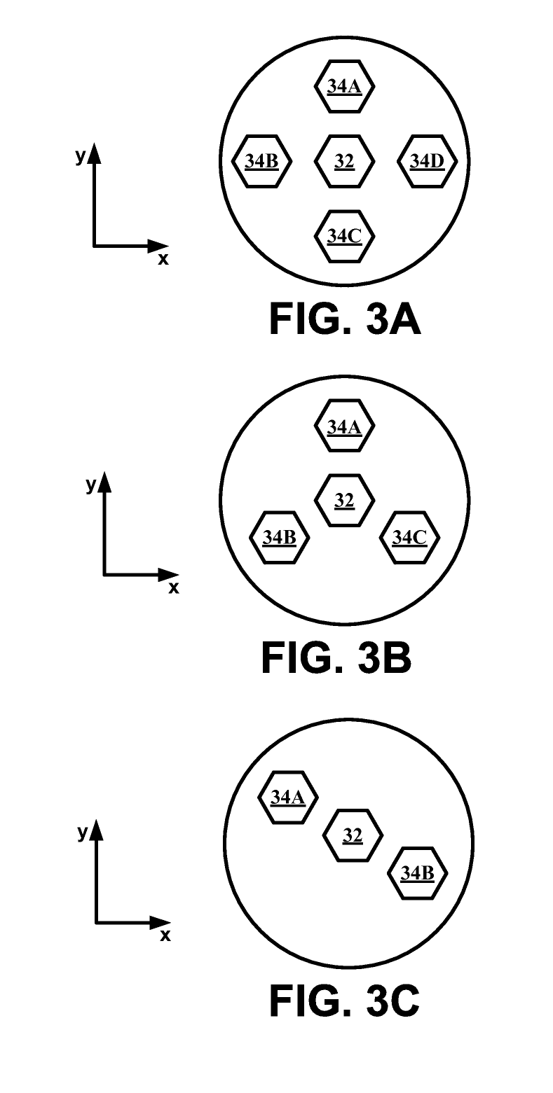

Each navigation sensor 34A-34D is spaced equidistant from the primary sensor 32. FIGS. 3A, 3B, and 3C illustrate example arrangements of navigation sensors in relation to the primary sensor 32. Specifically, FIG. 3A illustrates a first example configuration that includes four navigation sensors 34A-34B, FIG. 3B illustrates a second example configuration that includes three navigation sensors 34A-34C, and FIG. 3C illustrates a third example configuration 35C that includes two navigation sensors 34A-34B. In each of the example configurations, the primary sensor 32 and the navigation sensors are depicted in a transverse x-y plane. Other configurations, including configurations with more than four navigation sensors, are also possible.

Each of the navigation sensors 34A-34D measures a strength of the magnetic field and sends the processor component 40 a navigation signal that includes information indicative of the measured strength. Alternatively, the diagnostic device 30 may include an additional component (not shown) configured to combine the navigation signals to provide a combined navigation signal, which is then sent to the processor component 40.

Alternatively, one, or possibly more than one, of the navigation sensors 34A-34D do not measure the magnetic field. Measurements taken by such sensor(s) may provide additional information useable by the processor component 40 to determine whether primary sensor 32 is aligned over the implanted magnet and/or the magnet characteristic. For instance, one of the navigation sensors 34A-34D may be an accelerometer configured to measure an orientation of the diagnostic device 30 and/or the primary sensor 32. As another example, one of the navigation sensors 34A-34D may be a sensor configured to measure a distance from the diagnostic device 30 and/or the primary sensor 32 to the recipient's skin 16.

The interface module 36 includes one or more interactive components, such as buttons or switches, that allow clinician to interact with the diagnostic device 30. For example, the interface module 36 may include one or more buttons that the clinician depresses to cause the processor component 40 to determine whether the primary sensor 32 in the implanted magnet 20 are aligned and/or to determine the magnet characteristic. As another example, the clinician interacts with one or more buttons of the interface module 36 to select the magnet characteristic determined by the processor component 40. For instance, the clinician can interact with the interface module 36 to select strength, polarity, or identity (i.e., a name, part number or other designation of a particular magnet) as the magnet characteristic.

In one example, the interface module 36 also includes an external interface component configured to transmit data to and from the diagnostic device 30. For instance, the external interface component may include a wired interface component, such as the Universal Serial Bus (USB) interface or a proprietary wired interface, or a wireless interface component, such as a Wi-Fi interface or an RF interface. In this example, the interface module 36 receives one or more signals from the processor component 40 and transmits the received one or more signals to an external device via the external interface component.

Additionally, the interface module 36 receives one or more external signals from the external device, such as, perhaps, a request for data or an update to a program stored in the data storage 42. The interface module 36 sends the one or more external circuit signals to the processor component 40 for additional processing.

The output module 50 receives one or more of the characteristic signal, the alignment signal, and/or the direction signal from the processor component 40. The output module 50 includes a display component 52 and, in some examples, an optional speaker 54.

In response to receiving the characteristic signal, the output module 50 causes the display component 52 and/or the speaker 54 to output information indicative of the magnet characteristic. Similarly, in response to receiving the direction signal or the alignment signal, the output module 50 causes the display component 52 and/or the speaker 54 to output information indicative of the determined direction or of the primary sensor 32 and the implanted magnet 20 being aligned, respectively.

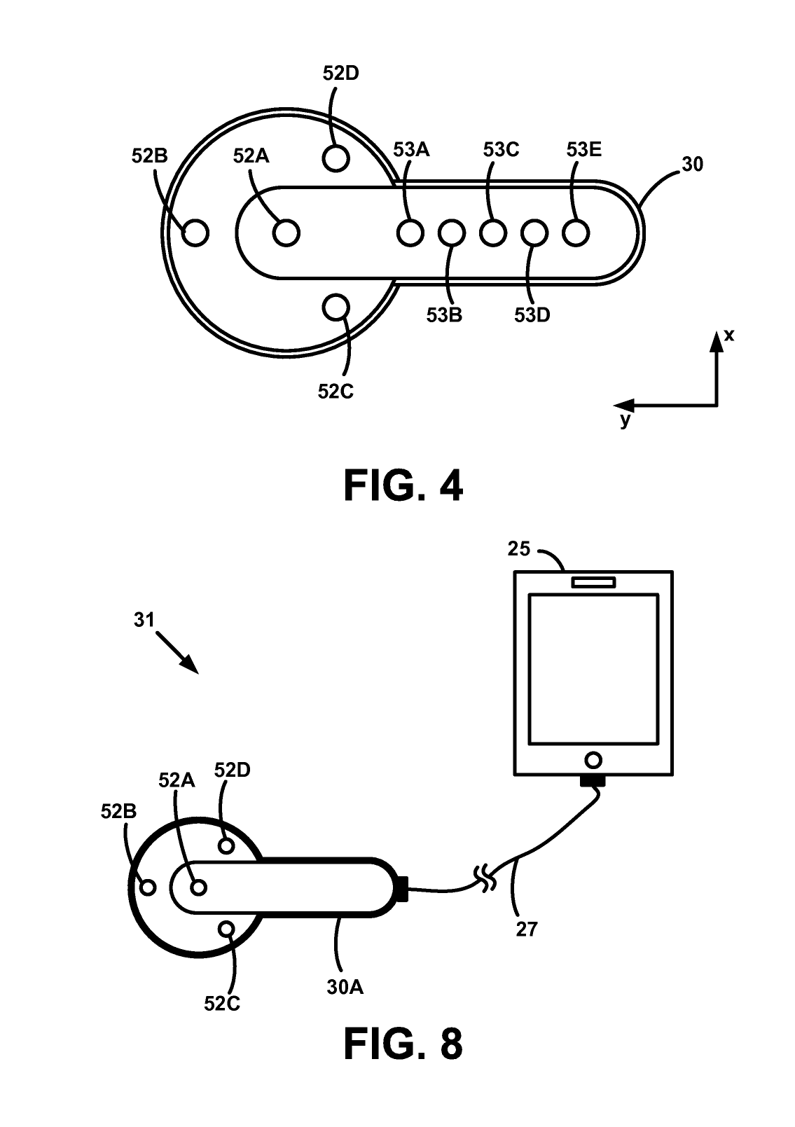

FIG. 4 is a top view of an example embodiment of the diagnostic device 30. In the illustrated example, a first LED array includes navigation LEDs 52A-52D, and a second LED array includes characteristic LEDs 53A-53D.

When lit, a first navigation LED 52A indicates that the primary sensor 32 is aligned over the implanted magnet 20. A second navigation LED 52B, a third navigation LED 52C, and a third navigation LED 52D correspond to a direction in which to move the diagnostic device 30 when the primary sensor 32 and the implanted magnet 20 are not aligned. To indicate the orientation of the sensors in the diagnostic device 30, the transverse x-y plane shown in FIGS. 3A-3C is also shown in FIG. 4.

The second LED array includes five characteristic LEDs 53A-53E. Each LED of the five characteristic LEDs corresponds to one of a plurality of magnet characteristics, such as a plurality of magnet strengths, or each LED corresponds to a particular magnet suitable for use as the external magnet 22.

In another example, the display component 52 includes a display device in lieu of the first LED array and/or the second LED array. In this example, the output module 50 causes the display component 52 to display information indicative of the recommended magnet and/or the determined direction on the display device. The output module 50 causes the display component 52 to display a graphical representation of an arrow on the display device, with the arrow pointing in the direction of the center the magnetic field (e.g., the direction in which the clinician should move the diagnostic device 32 align the primary sensor 32 and the implanted magnet 20).

As another example, the output module 50 causes the display component 52 to display an indication of the strength, the polarity, and/or the identification of the recommended magnet on the display device. The display device may include an LED display, an LCD display, the touchscreen, and/or any other display device or combination of display devices suitable for use in a diagnostic device 30. Other examples of graphical representations of the determined direction and/or of the magnet characteristic are also possible.

Returning to FIG. 2, the output module 50 also includes, in one example, the speaker 54. In this example, the output module 50 causes the speaker 54 to output an audible indication of the magnet characteristic, of the determined movement, and/or of primary sensor 32 and the implanted magnet 20 being aligned. For example, if the direction signal includes information indicative of the determined direction being to the left, the output module 50 causes speaker 54 to output an audio signal indicating that the clinician should move the diagnostic device 30 to the left, such as a recording of a person saying “move left.” As another example, the output module 50 causes the speaker 54 to output an audio signal that identifies the magnet characteristic, perhaps that identifies a recommended magnet by name, part number, etc. Other examples of audible indications are also possible.

The processor component 40 functions to receive and process signals from components of the diagnostic device 30, such as the primary sensor 32 and the navigation sensors 34A-34D. The processor component 40 also functions to generate output signals based on the processed signals, and to send the output signals to one or more components of the diagnostic device 30, such as the output module 50. To this end, the processor component 40 includes one or more processors and, perhaps, one or more additional components, such as an analog-to-digital converter.

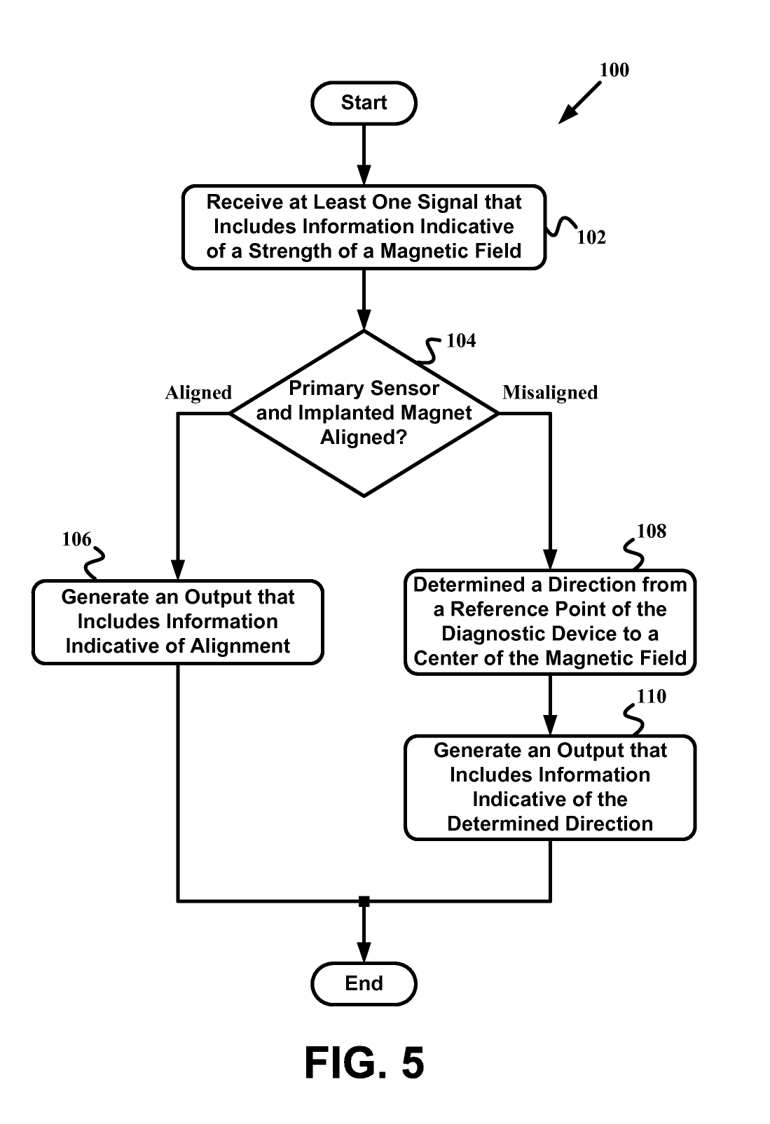

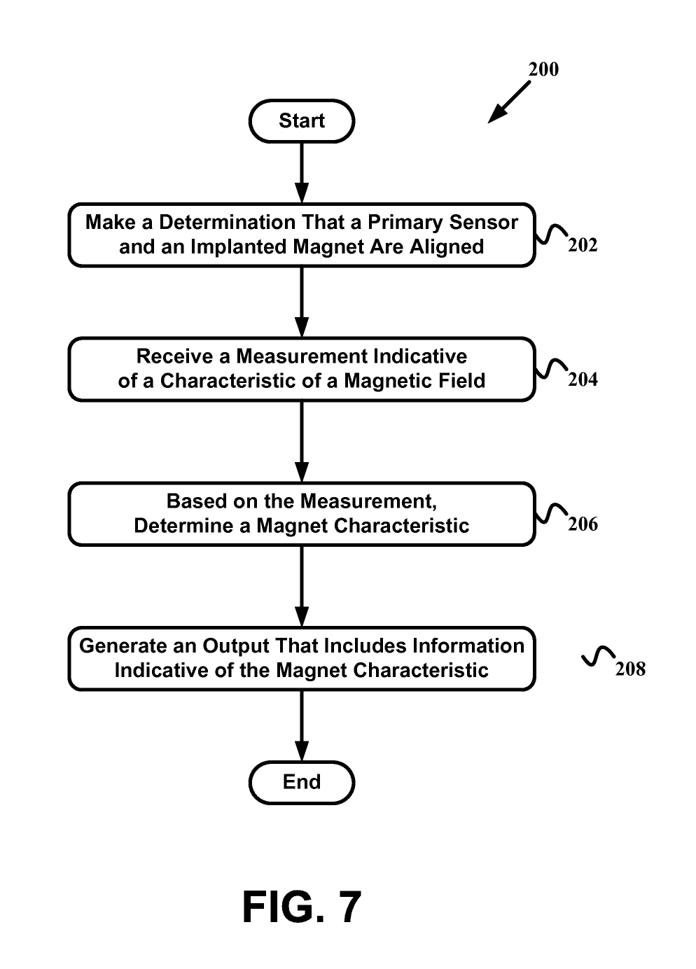

The processor component 40 also includes data storage 42. The data storage 42 includes any includes any type of non-transitory, tangible, computer readable media now known or later developed that is configurable to store program code for execution by the processor 44 and/or other data associated with the diagnostic device 30. The data storage 42 stores programs executable by the processor component 40 to determine the magnet characteristic, such as computer programs that cause the processor component 40 to perform one or more steps of the methods described herein with respect to FIGS. 5 and 7. In one example, the data storage 42 also stores one or more look-up tables.

In one example, the processor component 40 determines whether the primary sensor 32 and the implanted magnet 20 are aligned. FIG. 5 is a flow diagram of an example method 100 that the processor component 40 may perform to determine whether the primary sensor 32 and an implanted magnet 20 are aligned. While the method 100 and other methods herein are described with respect to the components of the implantable medical device depicted in FIG. 1 and the diagnostic device 30 depicted in FIG. 2, it is understood that other devices or systems can also implement the method 200.

At block 102, the method 100 includes receiving at least one signal that includes information indicative of a strength of a magnetic field. When performing the steps of block 102, the processor component 40 receives the navigation signals from the navigation sensors 34A-34D. Alternatively, in an example in which the primary sensor 32 configured to measure a vector of the magnetic field and the navigation sensors 34A-34D are not configured to measure a magnetic field (or are not included in the diagnostic device 30), the at least one signal includes at least one measurement signal generated by the primary sensor 32.

At block 104, the method 100 includes a decision point based on whether the primary sensor 32 and the implanted magnet 20 are aligned. The processor component 40 performs the steps of block 104 by processing the at least one signal and determining whether the information included in the at least one signal is indicative of the primary sensor 32 and the implanted magnet 20 being aligned.

In one example, the processor component 40 determines whether the primary sensor 32 and the implanted magnet 20 are aligned based on the information included in the navigation signal(s) received from the navigation module 34. As one example, the processor component 40 determines that the primary sensor 32 and the implanted magnet 20 are aligned if the measured characteristic of the magnetic field taken by each navigation sensor is the same or is substantially the same (e.g., the measured characteristics are each within a tolerance of one another). Other examples for determining that the primary sensor 32 and the implanted magnet 20 are aligned based on the information included in the navigation signal(s) are also possible.

In another example, the processor component 40 determines whether the primary sensor 32 and the implanted magnet 20 are aligned based on the information included in the primary signal(s) received from the primary sensor 32. In this example, the primary sensor 32 includes the vector of the magnetic field in the measurement signal, or the processor component 40 determines the vector based on one or more measurement signals received from the primary sensor 32. The processor component 40 determines that the primary sensor 32 and the implanted magnet 20 are aligned if the vector of the magnetic field is normal, or substantially normal, to the x-y plane of the primary sensor 32 depicted in FIG. 3. Other examples are also possible.

If the processor component 40 determined that the primary sensor 32 and the implanted magnet 20 are aligned, the method 100 includes generating an output that includes information indicative of the alignment, at block 106. When performing the steps of block 106, the processor component 40 generates the alignment signal and sends the alignment signal to the output module 50. In response to receiving the alignment signal, the output module 50 causes the display component 52 and/or the speaker 54 to output a visual indication or an audible indication, respectively, of the primary sensor 32 and the implanted magnet 20 being aligned.

If the processor component 40 determined that the primary sensor 32 and the implanted magnet 20 are misaligned at block 104, the method 100 includes determining a direction from a reference point of the diagnostic device to a center of the magnetic field, at block 108. In one example, the processor component 40 compares the characteristic of the magnetic field measured by each of the navigation sensors 34A-34D. For instance, if the measured characteristic is the strength of the magnetic field, the processor component 40 determines that the navigation sensor(s) measuring a greater strength of the magnetic field are closer to the center of the magnetic field. The processor component 40 determines the direction to the center of the magnetic field based on which of the navigation sensors 34A-34D are determined to be closer to the center of the magnetic field. Other examples for determining the direction based on information included in the one or more navigation signals are also possible.

In another example, the processor component 40 determines the direction based on the measured vector of the magnetic field. In this example, the processor component 40 determines the direction of the movement that results in the measured vector of the magnetic field being normal, or substantially normal, to the x-y plane of the primary sensor 32 depicted in FIG. 3. Other examples are also possible.

At block 110, the method 200 includes generating an output that includes information indicative of the determined direction. When performing the steps of block 110, the processor component 40 generates the direction signal and sends the direction signal to the output module 50. In response to receiving the alignment signal, the output module 50 causes the display component 52 and/or the speaker 54 to output a visual indication or an audible indication, respectively, of the direction in which to move the diagnostic device 30 in order to align the primary sensor 32 and the implanted magnet 20.

After completing the steps of block 110, the method 200 ends. The processor component 40 may repeat the method 100 until the primary sensor 32 and the implanted magnet 20 are aligned.

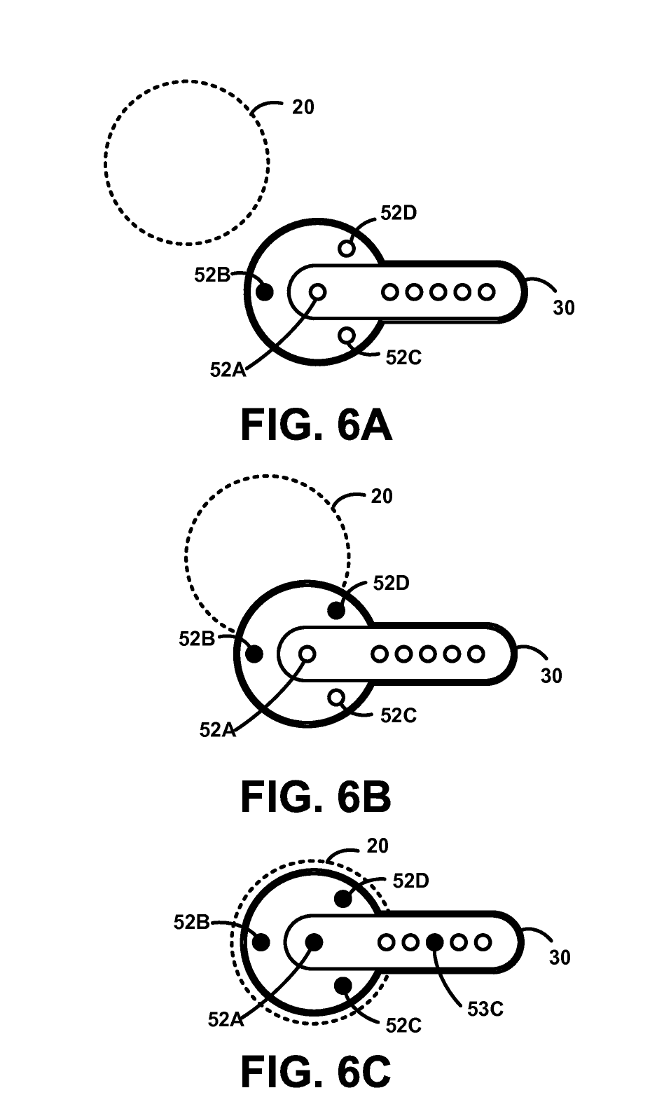

As a further description of the processor component 40 assisting a clinician in aligning the primary sensor 30 over the implanted magnet 20, FIGS. 6A, 6B, and 6C show aspects of the diagnostic device 30 in relation to the implantable magnet 20. While the diagnostic device 30, as depicted in FIG. 4, is depicted in each of FIGS. 6A, 6B, and 6C, it is understood that other embodiments of the diagnostic device 30 can be used as well.

In FIG. 6A, the processor component 40 determines that the primary sensor 32 and the implanted magnet 20 are misaligned. In response, the processor component 40 also determines that a direction from the primary sensor 32 (or another reference point of the diagnostic device 30) to the center of the magnetic field is to the left. The processor component 40 then generates a first direction signal that includes information indicative of the determined direction, and sends the first direction signal to the output module 50.

In response to receiving the first direction signal, the output module 50 causes the display component 52 to light the second navigation LED 52B, thereby providing an indication to the clinician to move the diagnostic device 30 to the left (the direction from the primary sensor 32 to the first navigation sensor 34A) in order to align the primary sensor 32 with the implanted magnet 20.

In FIG. 6B, the clinician has moved the diagnostic device 30 to the left based on the visual indication described in FIG. 6A. The processor component 40 again determines that the primary sensor 32 and the implanted magnet 20 are not aligned. In response to making this determination, the processor component determines that the center of the magnetic field is up and to the left. The processor component 40 then generates a second direction signal that includes information indicative of the determined direction, and sends the second direction signal to the output module 50.

In response to receiving the second direction signal, the output module 50 causes the display component 52 to light the second navigation LED 52B and the fourth navigation light 54D, thereby providing an indication to the clinician that, while the primary sensor 32 and the implanted magnet 20 are more closely aligned than in the example of FIG. 6A, the diagnostic device 30 needs to be moved up and to the left in order to align the primary sensor 32 with the implanted magnet 20.

In FIG. 6C, the clinician has moved the diagnostic device 30 up and to the left based on the visual indication depicted in FIG. 5B. The processor component 40 now determines that the primary sensor 32 and the implanted magnet 20 are aligned, and, in response, the processor component 40 generates the alignment signal, and send the alignment signal to the output module 50.

In response to receiving the alignment signal, the output module 50 causes the display component 52 to light the first navigation LED 52A. Additionally, as is illustrated in FIG. 6C, the output module 50 may cause the display component 52 to light the navigation LEDs 52B, 52C, and 52D as well. Other example light configurations are also possible.

The processor component 40 is also configured to determine a magnet characteristic for the external magnet 22. FIG. 7 is a flow diagram of an example method 200 that the processor component 40 may perform to determine the magnet characteristic.

At block 202, the method 200 includes determining that a primary sensor and an implanted magnet are aligned. When performing the steps of block 202, the processor component 40 performs the same or substantially similar steps as those described with respect to the method 100.

At block 204, the method 200 includes receiving a measurement indicative of a characteristic of a magnetic field. The processor component 40 receives one or more measurement signals from the primary sensor 32. As previously described, the primary sensor 32 includes information indicative of a measurement of a characteristic of the magnetic field in each measurement signal. In an example in which the processor component 40 receives more than one measurement signal from the primary sensor 32, the processor component 40 may determine a statistic, such as a mean, a median, or a mode, of the measurements included in the measurement signals.

At block 206, the method 200 includes determining, based on the measurement, a magnet characteristic. The processor component 40 processes the one or more measurement signals and, based on the information indicative of the measurement included in each of the one or more measurement signals, determines the magnet characteristic.

In one example, the processor component 40 accesses a first lookup table included in the data storage 42. The first lookup table includes a plurality of characteristics, with each magnet characteristic corresponding to one or more measured characteristics. The processor component 40 selects the characteristic corresponding to the measured characteristic. Alternatively, the processor component 40 may perform any algorithm, process, or method suitable for determining the magnet characteristic. Additionally, the processor component 40 may determine more than one magnet characteristic when performing the steps of block 206.

At block 208, the method 200 includes generating an output that includes information indicative of the magnetic characteristic. When performing the steps of block 208, the processor component 40 generates the characteristic signal, and sends the characteristic signal to the output module 50.

In response to receiving the characteristic signal, the output module 50 causes the display component 52 and/or the speaker 54 to provide a visual indication or audible indication, respectively, of the magnet characteristic. In the example depicted in FIG. 6C, for instance, the output module 50 causes the display component 52 to light the second characteristic light 53B, thereby providing an indication to the clinician of the magnet characteristic.

After performing the steps of block 208, the method 200 ends.

In the preceding examples, the processor component 40 determines whether the primary sensor 32 and the implanted magnet 20 are aligned prior to determining the magnet characteristic. In other examples, the processor component 40 determines whether the primary sensor 32 and the implanted magnet 20 are aligned in parallel to determining the magnet characteristic, perhaps by omitting step 202 of the method 200.

The diagnostic device 30 can also be implemented as a diagnostic system. FIG. 8 is a conceptual diagram of a diagnostic system 31. The diagnostic system 31 includes a computing device 25 and a sensor component 30A. In FIG. 8, the computing device 25 is illustrated as a tablet computer. In other examples, the computing device 25 is a laptop computer, a desktop computer, a smartphone, or any other computing device or combination of computing devices suitable for use in the diagnostic system 31.

The computing device 25 is connected to the sensor component 30A via a cable 27. In one example, the cable 27 is configured for use with a proprietary wired interface. In another example, the cable 27 is configured for use with standard wired interface, such as a USB interface. In yet another example, the computing device 25 and the sensor component 30A are connected via a wireless connection, such as a Wi-Fi connection or an RF connection.

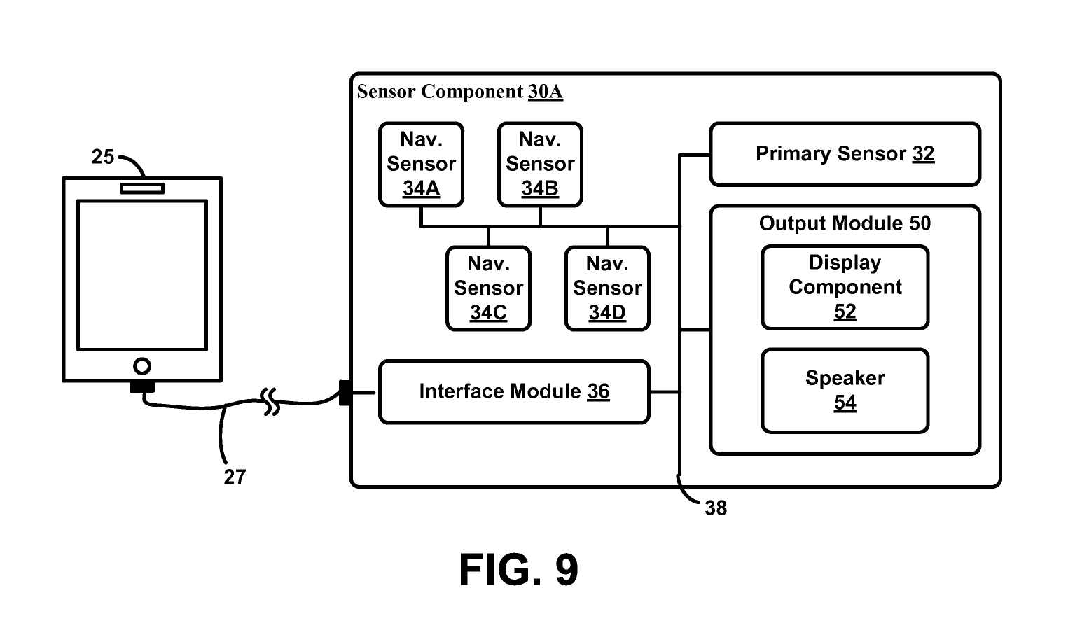

FIG. 9 is a simplified block diagram of the sensor component 30A depicted in FIG. 8. The sensor component 30A includes the primary sensor 32, the navigation sensors 34A-34D, the interface module 36, and the output module 50 described with respect to FIG. 2.

The diagnostic system 31 performs the functions of the diagnostic device 30, with the computing device 25 performing the functions described with respect to the processor component 40. The computing device 25 is also configured to display, and possibly store, information indicative of the determined direction and/or the magnet characteristic.

In the example illustrated in FIG. 9, the output module 50 includes the display component 52, and possibly the speaker 54. The computing device 25 sends the direction signal, the characteristic signal, and/or the alignment signal to the output module 50. In response to receiving one or more of these signals, the output module 50 causes the display component 52 and/or the speaker 54 to provide an output indicative of the information included in the received signal(s). In one example, an output provided by the provided by a component of the output module 50 supplements a visual output or an audible output provided by one or more components of the computing device 25.

In another example, the sensor component 30A does not include the output module 50. In this example, one or more components of the computing device 25 perform the functions described with respect to the output module 50.

While the diagnostic device 30 and the diagnostic system 31 are described as being used to determine external magnets to include in an external unit of an implantable medical device, the diagnostic device 30 and/or the diagnostic system 31 can be used in other applications in which a magnet is implanted in a recipient. For example, a clinician can use the diagnostic device 30 (or the diagnostic system 31) to assist in identifying an external magnet to include in an external apparatus, such as a cosmetic prosthesis.

With respect to any or all of the block diagrams, examples, and flow diagrams in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the scope of the invention being indicated by the following claims.

What is claimed is:

1. A diagnostic device comprising:
a primary sensor configured to measure a characteristic of a magnetic field generated by an implanted magnet, wherein the implanted magnet is implanted in a recipient;
an output component; and
a processor component configured to:
(i) receive one or more measurement signals from the primary sensor, wherein each measurement signal includes information indicative of a measured characteristic of the magnetic field;
(ii) based on the measured characteristic included in each of the one or more measurement signals, determine a magnet characteristic, wherein the magnet characteristic is indicative of a characteristic of an external magnet to include in an external apparatus that is configured to be magnetically attached to the recipient; and
(iii) cause the output component to output information indicative of the magnet characteristic.

2. The diagnostic device of claim 1, wherein:
the primary sensor includes a mechanical sensor and a magnetic object,
the mechanical sensor is configured to measure a force imparted by the magnetic field on the magnetic object, and
the measured characteristic is the measured force.

3. The diagnostic device of claim 1, wherein:
the primary sensor includes a magnetic field sensor configured to measure at least one of a strength of the magnetic field or a polarity of the magnetic field, and
the measured characteristic is one of the measured strength of the magnetic field or the measured polarity of the magnetic field.

4. The diagnostic device of claim 1, wherein:
the primary sensor includes a magnetic field sensor configured to measure a vector of the magnetic field, and
the measured characteristic is the measured vector.

5. The diagnostic device of claim 1, wherein, prior to determining the magnet characteristic, the processor component is configured to:
make a determination that the primary sensor and the implanted magnet are misaligned; and
responsive to making the determination, (i) determine a direction from a reference point of the diagnostic device to a center of the magnetic field, and (ii) cause the output component to output information indicative of the determined direction.

6. The diagnostic device of claim 5, wherein:
the primary sensor includes a magnetic field sensor configured to measure a vector of the magnetic field, and
the processor component is configured to determine that the primary sensor and the implanted magnet are aligned when the vector is substantially normal to a transverse plane of the primary sensor.

7. The diagnostic device of claim 6, wherein the output component comprises at least one of:
a display component, wherein, to output the information indicative of the determined direction, the output component is configured to cause the display component to display a visual indication of the determined direction; or
a speaker, wherein, to output the information indicative of the determined direction, the output component is configured to cause the speaker to output a sound that includes an audible indication of the determined direction.

8. The diagnostic device of claim 6, further comprising at least two navigation sensors configured to measure the magnetic field, wherein, to make the determination that the diagnostic device and the magnet are misaligned, the processor component is further configured to receive at least two navigation signals, wherein each of the at least two navigation signals (a) is generated by one of the at least two navigation sensors and (b) includes information indicative of a strength of the magnetic field.

9. The diagnostic device of claim 8, wherein each of the at least two navigation sensors are equidistant from the primary sensor.

10. The diagnostic device of claim 6, wherein:
the primary sensor includes a magnetic field sensor configured to measure a vector of the magnetic field,
the information indicative of the measured magnetic field for each measurement signal includes a measured vector of the magnetic field, and
prior to making the determination, the processor component is configured to receive a signal that includes information indicative of the measured vector, wherein each of the determination and the determined direction are based on the measured vector.

11. The diagnostic device of claim 1, wherein, to determine the magnet characteristic, the processor component is further configured to:
identify, based on the measured characteristic included in each of the one or more measurement signals, the external magnet from a plurality of magnets, wherein the magnet characteristic is an identification of the external magnet.

12. The diagnostic device of claim 1, wherein the output component comprises a display component, and wherein, to output the information indicative of the magnet characteristic, the output component is configured to cause the display component to display a visual indication of the magnet characteristic.

13. The diagnostic device of claim 1, wherein the magnet characteristic is one of a strength for the external magnet, a polarity for the external magnet, or an identification of the external magnet.

14. A method comprising:
receiving, by a component of a diagnostic device, a measurement indicative of a characteristic of a magnetic field generated by an implanted magnet, wherein the implanted magnet is included in an implantable unit of an implantable medical device;
based on the measurement, determining a magnet characteristic for an external magnet to include in an external unit of the implantable medical device; and
generating an output that includes information indicative of the magnet characteristic.

15. The method of claim 14, wherein the magnet characteristic is an identity of the external magnet, and wherein determining the magnet characteristic includes identifying, based on the measurement, the external magnet from a plurality of magnets.

16. The method of claim 15, wherein identifying the recommended magnet comprises:
comparing the measurements to a plurality of reference measurements, wherein each reference measurement corresponds to a magnet included in the plurality of magnets.

17. The method of claim 14, wherein the measurement comprises a measurement taken by a mechanical sensor included in the diagnostic device, wherein the measurement is a force imparted by the magnetic field on an object included in the diagnostic device.

18. The method of claim 14, wherein the measurement comprises a measurement taken by a magnetic field sensor included in the diagnostic device, and wherein the measurement is indicative of a vector of the magnetic field.

19. The method of claim 14, wherein the magnet characteristic is one of a strength for the external magnet or a polarity for the external magnet.

20. A system comprising:
a sensor device configured to measure a magnetic field generated by an implanted magnet, wherein the implanted magnet is included in an implantable unit of an implantable medical device; and
a computing device configured to:
receive a plurality of signals from the sensor device, wherein each signal includes information indicative of the measured magnetic field;
make a determination, based on a first set of signals included in the plurality of signals, of whether the sensor device and the implanted magnet are aligned or misaligned;
if the determination is that the sensor device and the implanted magnet are aligned, then (i) determine, based on a second set signals included in the plurality of signals, a magnet characteristic for an external magnet to include in an external unit of the implantable medical device, and (ii) generate a first output that includes information indicative of the magnet characteristic, wherein the second set of signals includes at least one signal; and
if the determination is that the sensor device and the implanted magnet are misaligned, then (i) determine, from at least two signals included the plurality of signals, a movement that will more closely align the sensor device over the implanted magnet, and (ii) generate a second output that includes information indicative of the movement.
